(19) United States
(12) Patent Application Publication
(10) Pub. No.: US 2026/0137951 A1
(43) Pub. Date: May 21, 2026

(54) HAND-HELD THERAPY DEVICE AND A CHARGING BASE

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/392,462

(22) Filed: Nov. 18, 2025

(30) Foreign Application Priority Data
Nov. 21, 2024 (CN) 2024228550548

(51) Int. Cl. A61N 5/06; A45D 34/00

(52) U.S. Cl. CPC A61N 5/0616; A45D 34/00; A45D 2034/005; A45D 2200/054; A45D 2200/205; A45D 2200/207; A61N 2005/0632; A61N 2005/0642; A61N 2005/0644; A61N 2005/0652

(57) ABSTRACT

Embodiments of the present invention disclose a hand-held therapy device and a therapy system. The hand-held device includes a housing having an end face, a side face, and an inclined treatment surface extending between the end face and the side face, a light-transmitting area on the treatment surface, and a phototherapy component disposed in a receiving cavity and aligned with the light-transmitting area to emit therapeutic light. A mounting structure on the housing detachably receives a care-product container. The device incorporates one or more stimulation elements and a battery with a wireless charging coil. The therapy system includes a charging base defining a slot, a second charging coil aligned with the first coil for inductive charging, and a shielding portion that covers the treatment surface during charging.

### TECHNICAL FIELD

The present invention relates to the field of personal care and therapeutic devices, and more particularly to a hand-held therapy device configured to provide phototherapy and optional multi-modal skin stimulation, as well as a therapy system including the hand-held therapy device and a charging base.

### BACKGROUND OF THE INVENTION

Hand-held therapeutic devices are widely used to deliver localized physiotherapy treatments such as vibration massage, microcurrent stimulation, phototherapy, and thermal therapy. These devices are typically designed as portable tools that can be easily maneuvered to different parts of the user's body. Despite their popularity, conventional devices suffer from various disadvantages that limit their convenience and usability.

The conventional hand-held phototherapy devices have rigid or flat treatment surfaces that do not conform well to the natural curvature of the human face. As a result, users must manually adjust their grip or the device angle during treatment, which reduces comfort and can lead to uneven light exposure and suboptimal therapeutic results.

These devices generally lack integrated structures for convenient use together with skincare products or cosmetic formulations. Users must separately apply serums, ointments, or creams, and then perform light therapy, which can be inconvenient and affect portability and ease of use. Many devices also fail to provide reliable attachment structures that allow a safe and stable connection to a cosmetic container.

Additionally, conventional phototherapy devices typically provide only a single type of treatment, such as light irradiation, without offering auxiliary stimulation functions such as vibration, heating, cooling, microcurrent, or electromagnetic stimulation. As a result, comprehensive treatment effects and improved user experience are not fully achieved.

Ergonomically, conventional therapeutic devices often do not provide a balanced or stable structure. Their housings typically include curved or inclined end surfaces, which prevent the device from standing upright. This reduces storage convenience and increases the risk of accidental falls when placed on flat surfaces. The inability to stand vertically also restricts the practical design of charging systems; many existing devices require angled cradles or precise alignment, making charging less intuitive for users.

Charging convenience is further reduced when the device must be inserted into dedicated cradles or docks, especially when electrical contact points are exposed, difficult to align, or subject to mechanical wear. Such designs can complicate routine use and reduce charging efficiency.

Accordingly, there is a need for an improved hand-held therapy device that integrates multiple stimulation elements, offers improved structural balance, supports greater ergonomic comfort, and provides simplified charging. There is also a need for a configuration that allows the device to stand upright independently using a stable end face, thereby improving storage practicality and providing a natural and more reliable interface with the charging base.

### OBJECTS OF THE INVENTION

Some of the objects of the invention are as follows:
An object of the present invention is to provide a handheld phototherapy device that features an angled light-transmitting surface designed to conform to the contours of a user's face or body, improving contact and comfort during skin treatment.
Another object of the invention is to integrate a mounting structure into the device housing that allows a skincare product container to be detachably attached, so that the user can apply the product and hold the device in a unified manner.
A further object is to design the light-transmitting surface and the internal LED array such that the width and distribution of light gradually taper from the top to the bottom, matching the shape of the face for more uniform illumination.
Yet another object is to include one or in combination of stimulation elements in the handheld device, such as vibration motors, heating or cooling elements, peltier modules, electromagnetic coils, micro-current electrodes, to provide additional therapeutic modalities alongside the light treatment.
An additional object of the invention is to provide a separate charging and storage base that wirelessly recharges the device. This base is configured with aligned inductive charging coils and a slot to receive the device in an upright orientation.
Another object is for the charging base to include a protective shield or cover that extends over the device's light-transmitting area when docked, preventing dust or scratches and blocking light leakage during charging.
A further object is to incorporate stability features in the charging base, such as a counterweight and an anti-slip mat, and to include guiding structures or alignment features that ensure the handheld device's charging coil is properly aligned with the base coil for efficient inductive charging.
Yet another object is to construct the device with a housing for the top cover and a main body that can be easily assembled. Internal support structures are provided to hold the battery and circuit board securely within the housing. The overall design is intended to be compact, lightweight, and easy to hold in various grip orientations for versatile use.

### SUMMARY OF THE INVENTION

According to a first aspect of the present invention, a hand-held therapy device is provided. The hand-held therapy device comprising: a housing having an end face, a side face, and a treatment surface extending between the end face and the side face, the treatment surface is inclined with respect to both the end face and the side face; a light-transmitting area disposed on the treatment surface; a phototherapy component disposed within the housing and facing the light-transmitting area, the phototherapy component is configured to emit light through the light-transmitting area for phototherapy treatment; and wherein the inclined treatment surface is shaped to conform to a facial contour of a user.

In one embodiment of the invention, the housing defines a receiving cavity for accommodating the phototherapy component and a light-transmitting opening communicating with the receiving cavity.

In one embodiment of the invention, the hand-held therapy device further comprising a light-transmitting plate embedded in the treatment surface and covering the light-transmitting opening, the light-transmitting plate forming the light-transmitting area.

In one embodiment of the invention, a surface of the light-transmitting plate protrudes outwardly from the treatment surface to facilitate close contact with the user's skin.

In one embodiment of the invention, the light-transmitting plate has an inverted-triangular shape with a width that gradually decreases from the end face toward the side face.

In one embodiment of the invention, the hand-held therapy device further comprising a mounting structure provided on the housing configured to detachably connect a container.

In one embodiment of the invention, the mounting structure comprises a slot formed at one end of the housing opposite the end face, the slot is configured to receive an insertion end of the container.

In one embodiment of the invention, the slot includes a pair of engaging protrusions arranged opposite each other on a sidewall of the slot to engage with the container.

In one embodiment of the invention, a clearance notch is defined between the engaging protrusions to prevent compression of a dispensing head of the container during installation.

In one embodiment of the invention, the housing comprises a top cover and a body that are detachably connected and together define the receiving cavity.

In one embodiment of the invention, the hand-held device further comprising one or more stimulation elements to provide one or more therapies.

In one embodiment of the invention, the one or more stimulation elements comprise a vibration element, a heating element, a cooling element, a Peltier element, an electromagnetic element, micro-current element.

According to a second aspect of the present invention, a therapy system is provided. The system comprising: a hand-held therapy device comprising a housing having an end face, a side face, and a treatment surface extending between the end face and the side face, the treatment surface is inclined with respect to both the end face and the side face; a phototherapy component configured to emit light for providing phototherapy treatment; and a charging base configured to detachably connect to the hand-held therapy device, wherein the charging base comprises: a housing defining a slot for receiving the hand-held therapy device; a shielding portion extending toward the hand-held therapy device and configured to cover the treatment surface during charging; and a second charging coil to connect with a first charging coil disposed within the hand-held therapy device to wirelessly charge the hand-held therapy device.

In one embodiment of the invention, the first charging coil is arranged adjacent to the end face of the hand-held therapy device, and the second charging coil is arranged adjacent to the bottom of the slot of the charging base.

In one embodiment of the invention, the charging base further comprises a second circuit board electrically connected to the second charging coil.

In one embodiment of the invention, the charging base comprises a charging interface electrically connected to the second circuit board and configured to receive external power.

In one embodiment of the invention, the charging base further comprises a counterweight disposed within the housing and spaced from the slot to enhance stability when the hand-held therapy device is inserted.

In one embodiment of the invention, the shielding portion is configured to prevent external light leakage during charging and to protect the light-transmitting area from dust or scratches.

In one embodiment of the invention, the hand-held therapy device and the charging base are shaped and dimensioned such that the first and second charging coils are magnetically aligned when the device is inserted into the slot, thereby improving charging efficiency.

In one embodiment of the invention, the charging base comprising a guiding structure configured to guide insertion of the hand-held therapy device into the slot and ensure proper positioning for charging.

According to a third aspect of the present invention, a hand-held therapy device is provided. The hand-held therapy device comprising: a housing having an end face, a treatment surface, and a receiving cavity; a phototherapy component disposed within the housing and oriented toward the treatment surface; a mounting structure configured to detachably receive a care product container containing a cosmetic liquid; a dispensing hole formed in the housing; and a liquid-dispensing structure comprising a liquid flow channel disposed within the housing and extending between the mounting structure and the dispensing hole, wherein the liquid flow channel is arranged to receive the cosmetic liquid from the care product container when the care product container is mounted to the mounting structure and to dispense the cosmetic liquid through the dispensing hole.

In one embodiment of the present invention, the hand-held therapy device further comprising: a second liquid flow channel disposed within the housing; and a selector switch positioned on a side wall of the housing and movable among at least three positions, wherein a first position closes both the liquid flow channel and the second liquid flow channel, a second position opens only the liquid flow channel to allow dispensing from a first compartment of the care product container, and a third position opens only the second liquid flow channel to allow dispensing from a second compartment of the care product container.

In one embodiment of the present invention, the liquid flow channel terminates at an outlet on the end face of the housing and the second liquid flow channel terminates at an outlet on the treatment surface of the housing to prevent cross-contamination between different cosmetic liquids.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “handheld therapy device” refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “head” or “phototherapy head” refer to a portion of the device coupled to the housing and configured to emit light toward the skin. The head may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 um), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain and inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a hand-held therapy device attached to a skincare container, in accordance with an embodiment of the present invention.

FIG. 2 is an exploded view showing the hand-held therapy device, a container, and a charging base, in accordance with an embodiment of the present invention.

FIG. 3 shows a perspective view of the hand-held therapy device alone, showing an end face, a side face, and a treatment surface, in accordance with an embodiment of the present invention.

FIG. 4 shows an exploded view of the treatment surface and a light-transmitting plate open, in accordance with an embodiment of the present invention.

FIG. 5 shows an exploded view of top portion of the hand-held therapy device, in accordance with an embodiment of the present invention.

FIG. 6 is a cross-sectional view of the hand-held therapy device, showing various components, in accordance with an embodiment of the present invention.

FIG. 7 shows the bottom perspective view of the hand-held therapy device, in accordance with an embodiment of the present invention.

FIG. 8 shows a cross-sectional view of another configuration of the hand-held therapy device, showing a flow channel in accordance with an alternate embodiment of the present invention

FIG. 9 shows a hand-held therapy device with two separate container and separate flow channels for dispensing two different cosmetic care solution, in accordance with an embodiment of the present invention.

FIG. 10 shows a top perspective view of a charging base for the hand-held therapy device in accordance with an embodiment of the present invention.

FIG. 11 shows a bottom perspective view of the charging base, in accordance with an embodiment of the present invention.

FIG. 12 is a cross-sectional view of the charging base in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a hand-held therapy device and a therapy system including a charging base. The features described in connection with any embodiment may be combined with other embodiments unless context indicates otherwise.

In one embodiment, the hand-held therapy device comprises a housing having an end face, a side face, and a treatment surface extending between the end face and the side face. The treatment surface is inclined relative to both the end face and the side face, thereby forming a contoured external profile. The inclination of the treatment surface allows the hand-held therapy device to make close and comfortable contact with the curvature of a user's facial region. The end face may be substantially planar, enabling the device to be stably supported in an upright orientation on a flat surface. This upright stability provides convenient storage and enhances alignment when the device is placed on a charging base.

The housing defines a receiving cavity configured to accommodate internal components. In one embodiment, the receiving cavity is formed by a body portion and a detachable top cover secured to the body by threaded engagement, snap structures, screw fasteners, or other mechanical connectors. The detachable connection allows convenient assembly, repair, or replacement of internal components.

A phototherapy component is disposed within the receiving cavity and faces a light-transmitting opening formed on the treatment surface. The phototherapy component includes one or more light-emitting devices such as LEDs, laser diodes, or other suitable lamps configured to emit therapeutic wavelengths, including visible or infrared light. A light-transmitting plate is embedded in the treatment surface and covers the light-transmitting opening to form a light-transmitting area. The plate is transparent or translucent and is made of material selected from one or polycarbonate, acrylic, quartz glass, or other optical material. In certain embodiments, the surface of the light-transmitting plate protrudes slightly outward from the treatment surface to improve conformal contact with a user's skin.

The light-transmitting plate has an inverted-triangular shape with a width that gradually decreases from the end face toward the side face. This geometry facilitates selective illumination of facial regions such as the cheeks, jawline, or periorbital areas.

In some embodiments, one or more stimulation elements are provided in the housing in addition to the phototherapy component. The stimulation elements are selected from a group consisting of, but not limited to, a vibration motor for mechanical stimulation, a heating or cooling element to deliver thermal therapy, a Peltier module for reversible temperature control, an electromagnetic stimulation module, and a micro-current module for applying low-level electrical stimulation.

The one or more stimulation elements are either arranged adjacent to the treatment surface or around the phototherapy component to deliver combined or sequential therapies. A control board within the receiving cavity is configured to operate the phototherapy component and stimulation elements independently or in combination according to user-selected modes.

The hand-held therapy device further includes a mounting structure for detachably connecting a container containing a cosmetic or therapeutic composition. In one embodiment, the mounting structure comprises a slot formed at an end of the housing opposite the end face. The slot is configured to receive the insertion end of the container. Opposing sidewalls of the slot further include a pair of engaging protrusions arranged to securely engage the container. A clearance notch is defined between the engaging protrusions to prevent pressure on the dispensing head of the container during installation, thereby avoiding accidental release of the stored composition.

The container dispenses its contents through a small outlet aligned with an applicator region of the device, permitting the device to simultaneously apply the composition and deliver phototherapy or other stimulation.

A first charging coil is disposed inside the housing, preferably adjacent to the end face. Positioning the coil near the end face facilitates magnetic alignment with a corresponding charging coil in the charging base when the device is placed upright within the base. The device further includes a power management module electrically coupled to the first charging coil to regulate wireless charging and control power delivery to internal therapy components.

In another embodiment, the invention provides a therapy system including the hand-held therapy device and a charging base. The charging base comprises a housing defining a slot configured to receive at least a portion of the hand-held therapy device. The slot is dimensioned to guide the device into an upright position with its end face resting against the bottom surface of the slot.

A second charging coil is disposed within the charging base and positioned adjacent to the bottom of the slot. When the device is inserted, the first and second charging coils are magnetically aligned to establish an inductive charging link. The charging base further includes a second circuit board coupled to the second charging coil for regulating charging operations.

The charging base further includes a shielding portion extending inward toward the treatment surface of the inserted device. The shielding portion is configured to cover the light-transmitting area when the device is docked, thereby preventing any light generated by accidental activation of the phototherapy component from escaping the base. The shielding portion also protects the treatment surface and the light-transmitting plate from dust, impact, and scratches during storage and charging.

Additionally, a guiding structure is provided within the slot to ensure proper alignment of the device and to prevent erroneous charging positions.

In certain embodiments, the charging base includes: a charging interface, such as a USB or DC port, for receiving external power; a counterweight disposed within the housing to enhance stability when the device is inserted; and indicator lights for showing charging or alignment status.

During treatment, the user places the treatment surface against the skin and activates one or more therapy modes via an interface disposed on the housing. The inclined geometry of the treatment surface allows improved conformity with facial contours, enhancing the uniformity of phototherapy and stimulation.

After use, the device is placed upright on its end face or inserted into the charging base. When inserted, the coils automatically align, and the shielding portion covers the treatment surface for safe charging.

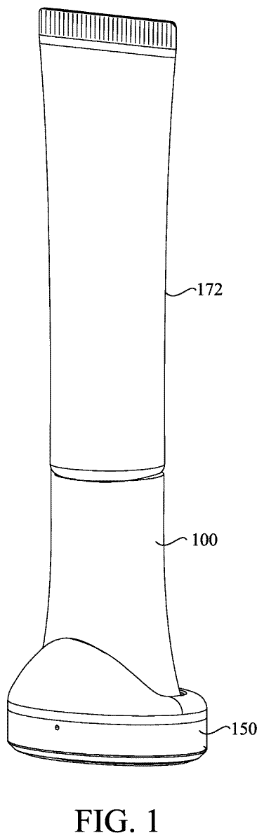

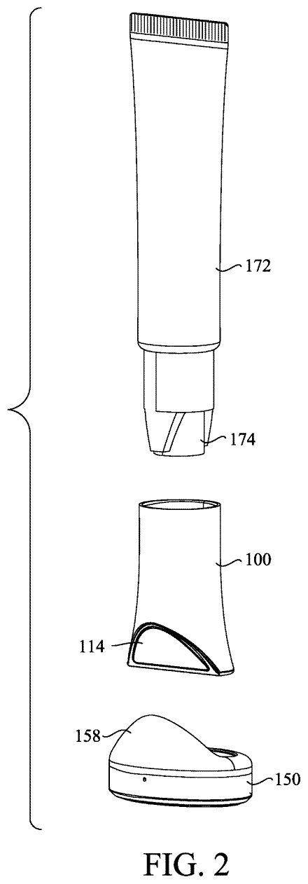

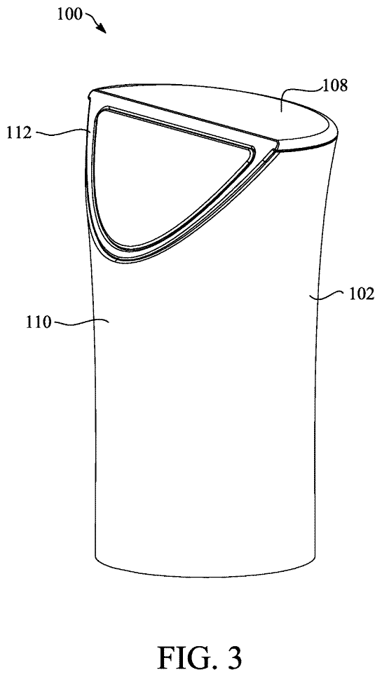

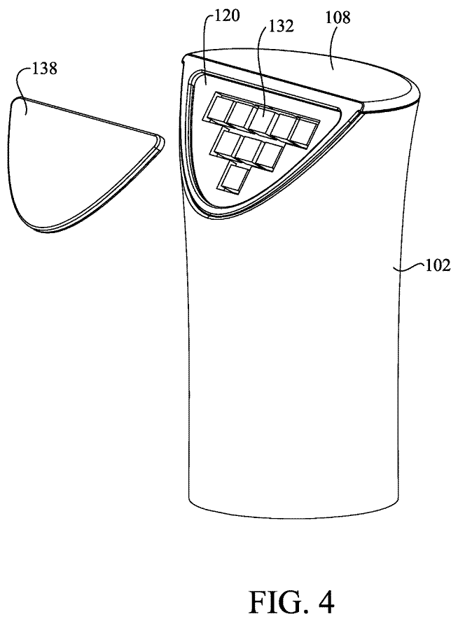

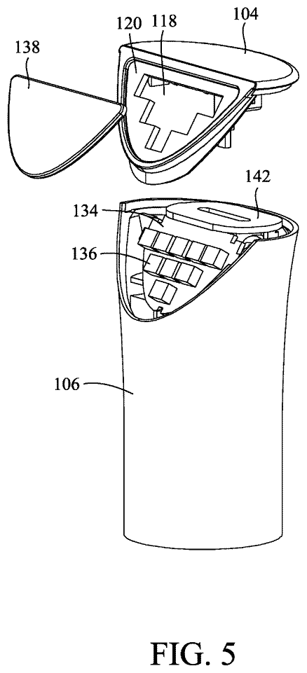

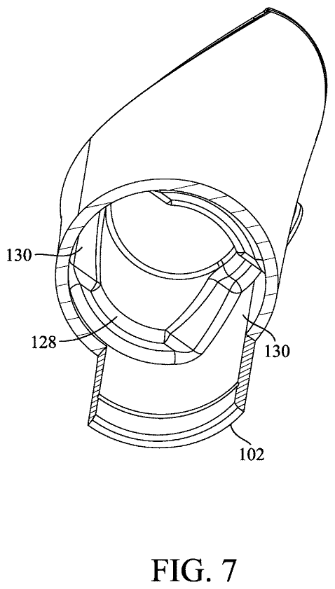

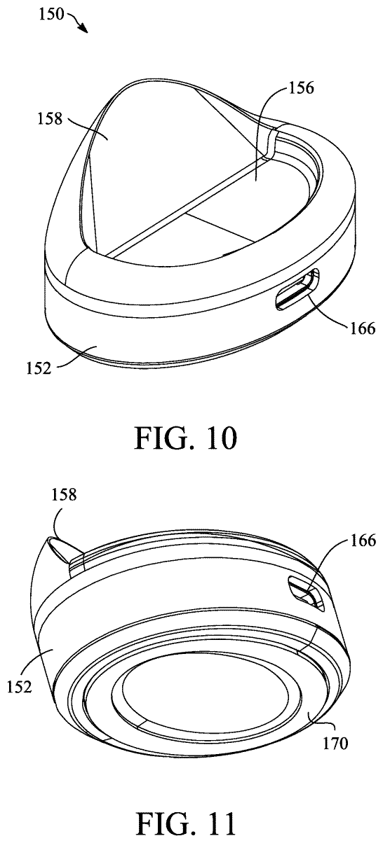

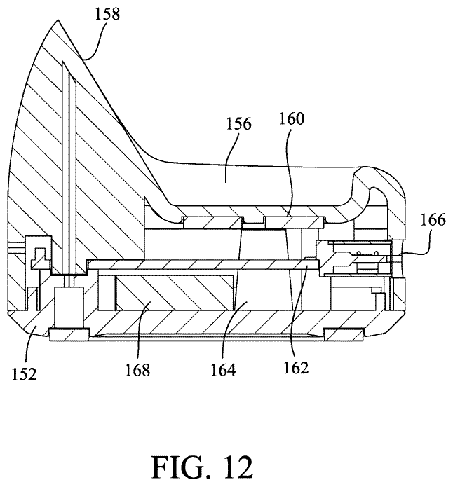

Embodiments of the present invention will now be described with reference to FIGS. 1 to 12.

Referring to FIGS. 1 to 7, the hand-held therapy device 100 includes a housing 102, a mounting structure 122, and a phototherapy component 132. The housing 102 has an end face 108, a side face 110, and a treatment surface 112. The treatment surface 112 extends from the end face 108 to the side face 110 and is arranged at an angle with respect to both the end face 108 and the side face 110. A light-transmitting area 114 is provided on the treatment surface 112, and light emitted by the phototherapy component 132 passes through the light-transmitting area 114 to achieve a phototherapy effect.

The mounting structure 122 is disposed on the housing 102 and detachably connects to a care product container 172, so that the user replaces care products according to different care needs. The care products are products used for skin care, beautification, or treatment, such as skin care products, cosmetics, and beauty-related ointments.

The phototherapy component 132 is disposed within the housing 102 and is positioned corresponding to the light-transmitting area 114. The phototherapy component 132 emits light outward through the light-transmitting area 114. After passing through the light-transmitting area 114, the light directly irradiates the user's skin or target area to achieve the desired phototherapy effect. Through light irradiation, skin cells are stimulated, blood circulation in the skin is improved, and metabolism is promoted, thereby achieving cosmetic or therapeutic effects.

In this application, the hand-held therapy device 100 adopts the treatment surface 112 formed at an angle with the end face 108 and the side face 110 of the housing 102. The angle of the treatment surface 112 conforms to the user's facial curve when the device is held. Correspondingly, the light-transmitting area 114 also conforms to the user's face. The user obtains a good phototherapy effect without additional hand adjustment when using the hand-held therapy device 100. This design improves the stability and comfort of the user's grip, and the user easily maintains contact between the hand-held therapy device 100 and the skin during use, ensuring that light is evenly distributed on the skin surface to obtain a more ideal phototherapy effect.

By providing the mounting structure 122 on the housing 102, the hand-held therapy device 100 combines phototherapy and care products and achieves integrated care. After using skincare products, cosmetics, or ointments, the user directly uses the hand-held therapy device 100 for assisted phototherapy, which promotes absorption of care ingredients and enhances the care effect. The mounting structure 122 also allows the user to conveniently combine the hand-held therapy device 100 with care products, thereby improving portability and storage of both the hand-held therapy device 100 and the care products.

Compared with conventional phototherapy devices, the hand-held therapy device 100 based on the technical solution of this application improves user comfort and portability.

In one embodiment, the housing 102 includes a receiving cavity 116 and a light-transmitting opening 118 communicating with the receiving cavity 116. The treatment surface 112 is provided with a mounting slot 120. The light-transmitting opening 118 is disposed on a groove wall of the mounting slot 120 and communicates with the receiving cavity 116. The phototherapy component 132 is disposed in the receiving cavity 116 and faces the light-transmitting opening 118.

The hand-held therapy device 100 also includes a light-transmitting plate 138 embedded in the mounting slot 120. The surface of the light-transmitting plate 138 is higher than the treatment surface 112 to form the light-transmitting area 114.

The light-transmitting plate 138 protects the internal phototherapy component 132 from external dust, moisture, and other influences, and allows the light to be emitted more evenly and softly through the light-transmitting process, avoiding direct light emission that irritates the skin and improving the uniformity and gentleness of the phototherapy effect.

The light-transmitting plate 138 increases the range of light illumination, similar to a lampshade. The mounting slot 120 improves the fastening strength of the light-transmitting plate 138 on the housing 102. In addition, the surface of the light-transmitting plate 138 being higher than the treatment surface 112 facilitates close contact between the light-transmitting plate 138 and the skin during use of the hand-held therapy device 100, so that the light-transmitting plate 138 forms a stable contact area when in contact with the skin, reduces scattering or deviation of light, and enables the light to be evenly and concentratively irradiated on the target skin area, thereby improving the uniformity and effectiveness of the phototherapy effect.

In other embodiments, the light-transmitting plate 138 is installed on the treatment surface 112 by a snap-fit, adhesive, or another mounting method. The surface of the light-transmitting plate 138 is flush with or lower than the treatment surface 112.

In one embodiment, the width of the light-transmitting plate 138 gradually decreases along a direction from the end face 108 to the side face 110. Specifically, the light-transmitting plate 138 has an inverted triangular shape. This design matches the shape of the housing 102 and optimizes the light projection area. The human face typically tapers from the forehead to the chin in a vertical direction, and the gradually decreasing width of the light-transmitting plate 138 fits this facial feature. This design allows the hand-held therapy device 100 to conform to facial contours in both the vertical and horizontal directions, avoids light gaps caused by facial narrowing, reduces blind spots, and makes the phototherapy effect more uniform across the entire face.

The phototherapy component 132 includes a lamp panel 134 and a plurality of lamp beads 136. The lamp panel 134 is arranged in parallel with the light-transmitting plate 138. The plurality of lamp beads 136 is arranged on a side of the lamp panel 134 facing the light-transmitting plate 138 and is inserted into the light-transmitting opening 118 to guide light effectively to the light-transmitting area 114.

In one embodiment, the plurality of lamp beads 136 is arranged in a decreasing order along the direction from the end face 108 to the side face 110. That is, the lamp beads 136 are arranged in an inverted triangular array corresponding to the light-transmitting plate 138.

The row-by-row decreasing arrangement of the lamp beads 136 adjusts the density of the lamp beads 136 according to the change in width of the light-transmitting plate 138, thereby ensuring uniform light distribution from the wider portion to the narrower portion of the light-transmitting plate 138. The light is emitted outward evenly through the light-transmitting plate 138, improving the phototherapy effect of the hand-held therapy device 100.

In other embodiments, the light-transmitting plate 138 is formed in other shapes, such as circular, rectangular, parallelogram, trapezoidal, or regular hexagonal. The layout of the lamp beads 136 on the lamp panel 134 is correspondingly adjusted according to the shape of the light-transmitting plate 138.

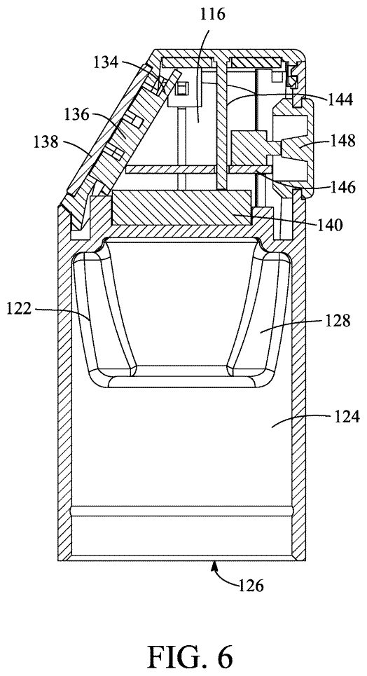

Referring to FIG. 6, in an embodiment, the device may comprise one or more stimulation components selected from a phototherapy component, a microcurrent component, a magneto-therapy component, a Peltier component, a heating and, a cooling component, and an ultrasonic wave therapy component. Each stimulation element may operate independently or in combination with the others to provide a multi-modal treatment experience.

In an embodiment, the stimulation component can be the microcurrent component configured to provide microcurrent stimulation therapy. The microcurrent component may include one or more electrodes disposed on the surface of the housing, such as on the end face or connecting surface, and electrically connected to the internal circuit board. The circuit board is configured to generate controlled low-level electrical pulses that are delivered through the electrodes to the user's skin. Such microcurrent stimulation aids in promoting circulation, improving cellular energy (ATP) production, and enhancing skin tone and elasticity. The microcurrent component may operate independently or concurrently with other stimulation elements such as the phototherapy, thermal, or ultrasonic modules to produce synergistic cosmetic and therapeutic effects.

In an embodiment, the stimulation component can be the heating and cooling component configured to provide controlled thermal therapy. The heating and cooling component may include a Peltier module, a resistive heating plate, or a thermoelectric component disposed beneath or adjacent to the light-transmitting plate. The thermal component is thermally coupled to the surface of the housing so that heat or coolness is effectively transferred to the user's skin. The control circuit regulates the direction and magnitude of current through the Peltier component to alternately produce heating or cooling effects.

In an embodiment, the stimulation component can be the magneto-therapy component that may include one or more electromagnetic coils or permanent magnets configured to generate a pulsed or static magnetic field to promote blood circulation and cellular metabolism.

The stimulation component can be the ultrasonic wave therapy component that may include one or more piezoelectric transducers adapted to emit ultrasonic vibrations in the range of 0.8-3 MHz to stimulate tissue regeneration, enhance transdermal absorption of skincare products, and relieve muscular tension.

In an embodiment, the phototherapy component provides optical stimulation using specific wavelengths of light, while the microcurrent component delivers controlled low-level electric currents through electrodes on the device surface. The heating and cooling components, including the thermoelectric (Peltier) component, regulate the surface temperature to deliver thermal therapy for soothing or tightening skin. All these components may be controlled individually or simultaneously through the circuit board and user interface, allowing the user to select desired therapy modes depending on treatment needs.

In one embodiment, the mounting structure 122 includes a first slot 124 disposed on the housing 102. The first slot 124 forms an insertion port 126 at one end of the housing 102 opposite the end face 108 to receive the care product container 172.

The first slot 124 allows insertion and connection between the housing 102 and the care product container 172. This structure enables quick and easy assembly and disassembly of the hand-held therapy device 100 and the care products, and the care product container 172 serves as a handle, facilitating movement and use of the hand-held therapy device 100. The insertion method reduces the lateral space occupied by the combination of the hand-held therapy device 100 and the care products and improves portability and storage.

In other embodiments, the mounting structure 122 is configured as a snap-on structure, hook structure, strap structure, threaded structure, or another structure.

In one embodiment, the first slot 124 and the receiving cavity 116 are separated by a partition. The care product container 172 and the phototherapy component 132 thus operate in independent spaces. Insertion and removal of the care product container 172 do not interfere with the operation of the phototherapy component 132, thereby ensuring stability and service life of the phototherapy component 132.

In other embodiments, the receiving cavity 116 and the first slot 124 are in communication.

In one embodiment, the mounting structure 122 further includes two engaging protrusions 128 protruding from a sidewall of the first slot 124. The two engaging protrusions 128 are arranged opposite each other to engage with a dispensing end of the care product container 172. The two engaging protrusions 128 are spaced apart to form a clearance notch 130 on the sidewall of the first slot 124 to avoid obstructing a dispensing head 174 of the care product container 172. The dispensing head 174 (or dispensing nozzle) is the part of the care product container 172 used for liquid dispensing and is an opening structure of the container. The dispensing head 174 controls the dispensing of care products such as skincare products, cosmetics, or ointments and ensures that liquid is accurately and appropriately discharged from the container for application by the user.

The two engaging protrusions 128 tightly engage the liquid outlet region of the care product container 172, preventing the container from shaking or loosening during use and ensuring a stable connection between the hand-held therapy device 100 and the care product container 172. This stable connection improves operating smoothness and enhances safety and user experience.

Because the gap between the engaging protrusions 128 forms the clearance notch 130, the care product container 172 avoids compression of the dispensing head 174 during installation. This prevents accidental leakage of the care product due to squeezing of the dispensing head 174 and prevents damage to the dispensing head 174, maintaining the normal dispensing function of the care product container 172.

In one embodiment, the housing 102 includes a top cover 104 and a main body 106 that are detachably connected and enclose the receiving cavity 116. The top cover 104 has the end face 108 and the treatment surface 112. The main body 106 is elongated and has the side face 110 and the first slot 124. The top cover 104 and the main body 106 are detachably connected and together form the receiving cavity 116 for mounting the phototherapy component 132.

The elongated main body 106 improves grip and facilitates assembly of the hand-held therapy device 100 with the care product container 172. The detachable connection between the top cover 104 and the main body 106 allows the receiving cavity 116 to be opened independently. This structure facilitates installation, inspection, and replacement of the phototherapy component 132, simplifies maintenance of the hand-held therapy device 100, and simplifies initial assembly of the hand-held therapy device 100.

In other embodiments, the top cover 104 and the main body 106 are detachably connected by screw connection, threaded connection, snap-fit connection, plug-in connection, adhesive bonding, or another connecting mechanism.

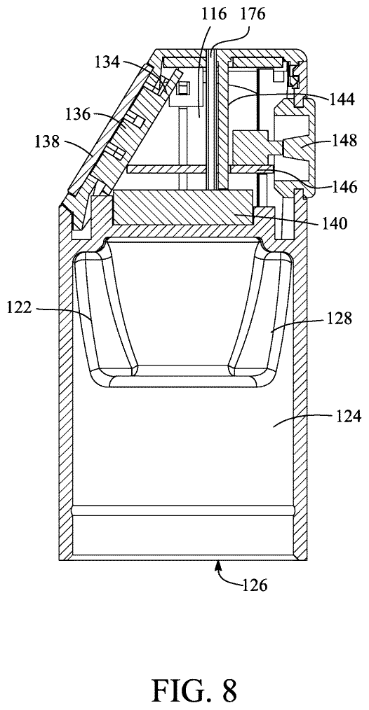

In an alternate embodiment of the present invention, the hand-held therapy device 100 further includes a liquid-dispensing structure that enables a cosmetic liquid contained in a care product container 172 to be delivered directly through the device. Referring to FIG. 8, the top cover 104 of the housing 102 is formed with a dispensing hole, and an internal liquid flow channel 176 is provided within the housing 102. The liquid flow channel 176 is in fluid communication with the dispensing hole and extends toward a mounting structure configured to receive the care product container 172. When the care product container 172 is installed, the outlet of the container aligns with the inlet of the liquid flow channel 176, allowing liquid to enter the channel and be dispensed through the dispensing hole.

The liquid flow channel 176 may be defined by walls integrally formed on the inner surface of the main body 106, or by a separate conduit fixed within the receiving cavity 116. The liquid flow channel 176 may be dimensioned to regulate liquid flow, prevent leakage, and maintain separation between the liquid path and internal components such as the phototherapy component 132, the first wireless charging coil 142, the battery 140, and the first circuit board 146. In some embodiments, a flow-control structure such as a one-way valve, a flexible diaphragm, or a metering element may be provided near the dispensing hole to permit user-controlled or pressure-activated dispensing.

This integrated structure allows the cosmetic liquid to be dispensed directly toward the treatment surface 112 while the device is operating. As a result, skincare liquid can be applied precisely at the location where phototherapy or stimulation is performed, improving absorption efficiency and synergizing with the emitted phototherapy light passing through the light-transmitting area 114. Because the dispensing hole is positioned away from the light-transmitting area 114, liquid does not interfere with optical output, ensuring stable light emission and protecting the optical components.

The combination of the care product container 172, the liquid flow channel 176, and the dispensing hole provides several advantages. First, it enables one-handed operation, as the user does not need to handle a separate cosmetic bottle. Second, it provides precise and controlled liquid application exactly where treatment is delivered, creating a more efficient and convenient skincare process. Third, the internal routing of liquid minimizes accidental spills and maintains a clean and compact device structure. Additionally, the separation between the liquid path and the electronic components ensures operational safety, prevents short-circuiting, and enhances the overall durability of the hand-held therapy device 100.

This embodiment may be used independently or in combination with any of the foregoing embodiments, including the inclined treatment surface, detachable mounting structure for the care product container 172, the shielding portion 158 of the charging base 150, or the wireless charging features.

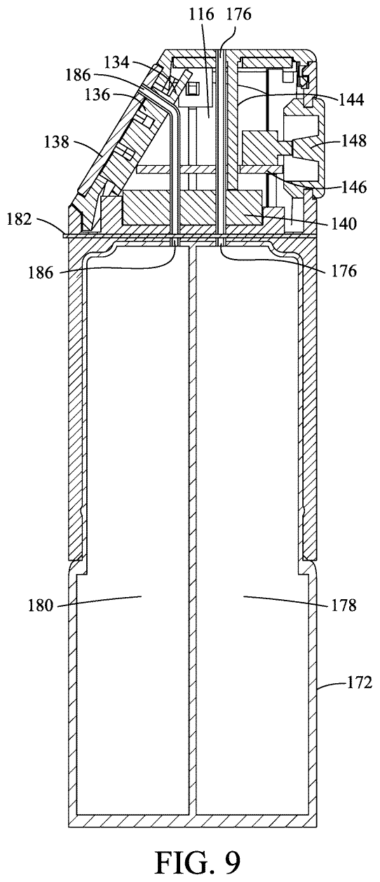

In another embodiment, illustrated in FIG. 9, the hand-held therapy device 100 is configured to dispense two different cosmetic or treatment liquids through a dual-channel delivery arrangement. In this embodiment, the liquid container 172 is constructed as a dual-compartment container having a first compartment 178 and a second compartment 180, each independently sealed to store different liquid or gel formulations for separate therapeutic applications. The hand-held therapy device 100 incorporates two isolated liquid flow paths that correspond to the two compartments. Specifically, the first compartment 178 is in fluid communication with the liquid flow channel 176, while the second compartment 180 is in fluid communication with a second liquid flow channel 186. To prevent cross-contamination between the two formulations, the liquid flow channel 176 and the second liquid flow channel 186 are arranged to terminate on different dispensing surfaces of the device. In the illustrated configuration, the liquid flow channel 176 opens on the end face 108 of the housing 102, whereas the second liquid flow channel 186 opens on the treatment surface 112. This spatial separation allows one formulation to be applied from the end face 108 while the other formulation can be applied from the inclined treatment surface 112, ensuring that the surface used for one liquid does not become contaminated by the other.

The liquid flow channel 176 and the second liquid flow channel 186 may be integrally formed within the housing 102 or may be provided as internal conduits arranged within the receiving cavity 116 without interfering with components such as the phototherapy component 132, the battery 140, the first circuit board 146, or the first wireless charging coil 142. To enable the user to selectively dispense liquid from either the first compartment 178 or the second compartment 180, or to prevent dispensing entirely, the hand-held therapy device 100 is equipped with a selector switch 182 positioned on a side wall of the housing 102. The selector switch 182 includes three switch positions. When the switch 182 is placed in a first position, both liquid flow channels 176 and 186 remain closed, blocking dispensing from both compartments. When the selector switch 182 is placed in a second position, only the first liquid flow channel 176 is opened, allowing liquid from the first compartment 178 to be dispensed through the outlet on the end face 108. When the selector switch 182 is moved to a third position, only the second liquid flow channel 186 is opened, allowing liquid from the second compartment 180 to dispense through the outlet on the treatment surface 112. The selector switch 182 may employ a rotary valve, sliding gate, or other sealing mechanism to reliably close the non-selected channel while permitting controlled flow through the selected one.

Although FIG. 9 illustrates a single liquid container 172 having two internal compartments, the present embodiment is not limited to this structure. In alternative implementations, two independent liquid containers may be mounted to the mounting structure of the device, with each container supplying one of the liquid flow channels 176 and 186. In such a configuration, the selector switch 182 operates in the same manner to permit dispensing from either selected container or to block dispensing entirely. This multi-channel dispensing design provides significant advantages, enabling users to conveniently apply two different cosmetic or therapeutic formulations using a single handheld device while maintaining complete separation of the two liquids. By discharging each formulation from a different dispensing surface, specifically, the end face 108 and the treatment surface 112, the likelihood of contaminating one application surface with another formulation is minimized. This arrangement also eliminates the need for users to switch between separate applicators, thereby improving convenience, hygiene, and operational efficiency.

In an embodiment of the present ion, the phototherapy component and the one or more stimulation elements are provided on the end face 108 in addition to the treatment surface 112. The user can use the phototherapy component and the one or more stimulation element on the end face 108 when using the liquid dispensed from the flow channel 176 provided on the end face 108.

Referring to FIGS. 1 to 12, the present invention also provides a therapy system that includes a charging base 150 and the hand-held therapy device 100. The charging base 150 is detachably connected to the hand-held therapy device 100 and charges the hand-held therapy device 100. The specific structure of the hand-held therapy device 100 is as described in the preceding embodiments.

By providing the charging base 150, the user places the hand-held therapy device 100 directly onto the charging base 150 for charging. This arrangement delivers a stable power source to the hand-held therapy device 100, maintains sufficient operating power, and enables the device to function independently without relying on an external wired power supply, thereby improving overall usability.

Furthermore, since the therapy system applies all technical features of the above embodiments, it achieves at least all corresponding beneficial effects, which are not repeated here.

In some embodiments, the charging base 150 includes a base housing 152 provided with a second slot 156 and a shielding portion 158. The second slot 156 receives the hand-held therapy device 100, and the shielding portion 158 extends toward the hand-held therapy device 100 to cover the surface of the light-transmitting area 114. This configuration prevents the light-transmitting area 114 from being exposed during charging, thereby reducing dust accumulation, contamination, scratches, and other potential damage, maintaining the cleanliness and integrity of the light-transmitting area 114, and improving the service life of the hand-held therapy device 100.

During charging, the shielding portion 158 blocks the light-transmitting area 114, preventing stray light from being emitted toward the user or the surroundings, and eliminating possible irritation to the skin or eyes. This structure enhances user safety, particularly in low-light environments. In alternative embodiments, the hand-held therapy device 100 is simply placed on the charging base 150 without insertion.

In some embodiments, the hand-held therapy device 100 includes a battery 140 and a first wireless charging coil 142 disposed in the receiving cavity 116. The battery 140 is electrically connected to both the phototherapy component 132 and the first wireless charging coil 142. The charging base 150 includes a second wireless charging coil 160. The second wireless charging coil 160 induces electromagnetic coupling with the first wireless charging coil 142 to wirelessly charge the battery 140.

Wireless charging is achieved through electromagnetic induction between the first wireless charging coil 142 and the second wireless charging coil 160. The user only needs to place the hand-held therapy device 100 onto the charging base 150 to initiate charging, eliminating the need for manual cable connection or removal. This simplifies the operation and improves convenience. Furthermore, due to the wireless charging design, the housing 102 of the hand-held therapy device 100 does not require an external charging port, reducing openings, improving sealing and waterproofing, and extending service life.

Since the first wireless charging coil 142 is electrically connected to the battery 140, the hand-held therapy device 100 also supports charging through other wireless charging devices, thereby expanding available charging methods.

In some embodiments, the first wireless charging coil 142 is positioned within the receiving cavity 116 adjacent to the end face 108, and the second wireless charging coil 160 is positioned within the base housing 152 adjacent to the bottom of the second slot 156. This alignment ensures that, when the hand-held therapy device 100 is placed in the charging base 150, the two wireless charging coils accurately correspond to each other, improving electromagnetic induction efficiency and reducing energy loss.

Additionally, the hand-held therapy device 100 includes a first circuit board 146 disposed in the receiving cavity 116, the first circuit board 146 having a first clearance hole. The inner surface of the top cover 104 includes a limiting post 144 that passes through the first clearance hole and abuts against the battery 140. The side of the battery 140 opposite the limiting post 144 abuts against the main body 106. This structure fixes and supports the battery 140, preventing displacement or vibration during use or transportation, ensuring operational safety, and improving overall durability. The limiting post 144 also stabilizes the first circuit board 146.

In other embodiments, the first circuit board 146 and the battery 140 are spaced apart to avoid direct contact, preventing pressure transmission from the battery 140 to the first circuit board 146 during vibration, reducing wear or potential damage, and extending device lifespan.

Alternatively, a shock-absorbing pad is arranged between the first circuit board 146 and the battery 140. The inner surface of the top cover 104 still carries the limiting post 144, which abuts against the side of the first circuit board 146 opposite the battery 140. This configuration stabilizes both components while attenuating vibration transmission, providing additional protection. The shock-absorbing pad is formed of rubber, foam, or similar elastic material.

In some embodiments, the hand-held therapy device 100 includes a button 148 exposed on the housing 102 and electrically connected to the first circuit board 146 to control the functions of the hand-held therapy device 100. The button 148 is configured as a touch button or a mechanical button.

In some embodiments, the charging base 150 includes a second circuit board 162 disposed in the base housing 152. The second circuit board 162 includes a second clearance hole. The inner surface of the base housing 152 includes an abutment post 164 that passes through the second clearance hole and abuts the surface of the second wireless charging coil 160. This arrangement fixes the second wireless charging coil 160, prevents vibration or shifting during use, maintains accurate alignment with the first wireless charging coil 142, and ensures stable and efficient charging. The abutment post 164 also separates the second wireless charging coil 160 from the second circuit board 162, preventing interference or wear and improving reliability.

In some embodiments, the charging base 150 includes a charging interface 166 electrically connected to the second circuit board 162 to supply power to the charging base 150. The charging interface 166 adopts a standard interface such as USB, Type-C, or DC, enabling convenient connection to external power supplies, including adapters, power banks, or computers, thereby improving versatility and user convenience.

In some embodiments, the charging base 150 includes a counterweight 168 arranged within the base housing 152 away from the second slot 156. The counterweight 168 increases the weight of the charging base 150, enhancing stability when the hand-held therapy device 100 is inserted or removed, preventing tipping, and ensuring smooth operation.

In some embodiments, the charging base 150 includes an anti-slip mat 170 disposed on the side of the base housing 152 opposite the hand-held therapy device 100. The anti-slip mat 170 increases friction against the supporting surface, prevents sliding, and improves charging stability. It also protects the supporting surface and provides cushioning.

In certain embodiments, the present invention further provides a method of delivering combined light-based therapy and supplemental stimulation using the hand-held therapy device described earlier. During use, a user grasps the housing of the device such that the inclined treatment surface can be brought into contact with, or positioned closely adjacent to, a target region of the skin. The housing includes the end face, the side face, and the treatment surface extending at an angle between them, allowing ergonomic handling and precise placement along various facial contours. The treatment surface includes a light-transmitting area behind which the phototherapy component is arranged within the receiving cavity of the housing. When the device is actuated, the phototherapy component emits light through the light-transmitting area toward the skin to deliver phototherapy to the selected region.

The device may additionally incorporate one or more stimulation elements that operate simultaneously with the phototherapy treatment. These stimulation elements may include vibration generators, thermal regulation modules, microcurrent electrodes, or other sensory stimulation components positioned adjacent to or integrated with the treatment surface. During operation, the device may apply one or more of these stimulations based on predetermined programs or user-selected settings. The combined effect of light emission and additional stimulation enables improved cosmetic and therapeutic outcomes.

The device may further include a mounting structure configured to receive a care-product container. When a container is mounted, the device can dispense or apply a skin-treatment formulation in coordination with light therapy and stimulation. The mounting structure may be implemented using plug-in, threaded, snap-fit, or magnetic engagement elements to allow secure yet detachable coupling of the container. As the user moves the device along the skin, the care product may be applied concurrently with phototherapy or other stimulation functions, enabling more efficient skin absorption and combined treatment benefits.

The device may also include sensors configured to detect skin contact, orientation, or proximity. Based on sensor feedback, the device can automatically adjust the operating intensity of the phototherapy component or stimulation elements to ensure safe and optimized treatment conditions. In certain embodiments, a temperature-control element maintains the temperature of the treatment surface within a selected therapeutic range during use, ensuring comfort and consistency throughout the treatment cycle.

The present invention also provides a method for charging the hand-held therapy device using the cooperating charging base. To initiate charging, the user positions the device so that the end face is received within a slot formed in the housing of the charging base. The geometry of the end face enables the device to rest stably in an upright orientation when inserted into the slot. As the device is placed in this orientation, a first charging coil housed within the device becomes aligned with a second charging coil located in the charging base. Once alignment is achieved, wireless power transfer occurs, allowing energy to be inductively transferred from the charging base to the device battery.

The charging base may include a shielding portion that extends toward the device when the device is seated in the slot. This shielding portion partially surrounds or covers the treatment surface during charging, thereby protecting the light-transmitting area from dust, external light exposure, or accidental contact. The shielding portion also helps the device maintain a stable position while charging.

In some embodiments, the device may detect when it is correctly positioned in the charging base and automatically switch to a charging or low-power standby mode. Furthermore, the physical cooperation of the end face and the slot ensures reliable alignment of the charging coils and prevents tipping or unstable placement during the charging process.

The invention finds industrial application in the cosmetics, dermatology, personal care, and wellness device industries, where compact, multi-modal treatment tools are in high demand. The device may be mass-produced using conventional plastic moulding, electronic assembly, and consumer-grade manufacturing processes, making it suitable for large-scale commercial production. It may be marketed as a premium home-use skincare tool, professional aesthetician device, or integrated therapy instrument for salons, spas, dermatology clinics, and beauty centres. Its ergonomic design, modular stimulation components, and compatibility with skincare product containers offer significant advantages for both consumer and professional markets.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A hand-held therapy device, comprising:
a housing having an end face, a side face, and a treatment surface extending between the end face and the side face, the treatment surface is inclined with respect to both the end face and the side face;
a light-transmitting area disposed on the treatment surface;
a phototherapy component disposed within the housing and facing the light-transmitting area, the phototherapy component is configured to emit light through the light-transmitting area for phototherapy treatment; and
wherein the treatment surface is inclined and shaped to conform to facial contour of a user.

2. The hand-held therapy of claim 1, wherein the housing defines a receiving cavity for accommodating the phototherapy component and a light-transmitting opening communicating with the receiving cavity.

3. The hand-held therapy device of claim 2 further comprising a light-transmitting plate embedded in the treatment surface and covering the light-transmitting opening, the light-transmitting plate forming the light-transmitting area.

4. The hand-held therapy device of claim 3, wherein a surface of the light-transmitting plate protrudes outwardly from the treatment surface to facilitate close contact with the user's skin.

5. The hand-held therapy device of claim 3, wherein the light-transmitting plate has an inverted-triangular shape with a width gradually decreasing from the end face toward the side face.

6. The hand-held therapy device of claim 1, further comprising a mounting structure provided on the housing configured to detachably connect a container.

7. The hand-held therapy device of claim 6, wherein the mounting structure comprises a slot formed at one end of the housing opposite the end face, the slot is configured to receive an insertion end of the container.

8. The hand-held therapy device of claim 7, wherein the slot includes a pair of engaging protrusions arranged opposite each other on a sidewall of the slot to engage with the container.

9. The hand-held therapy device of claim 1, further comprising one or more stimulation elements to provide one or more therapies, wherein the one or more stimulation elements comprise a vibration element, a heating element, a cooling element, a Peltier element, an electro-magnetic element, micro-current element.

10. A therapy system, comprising:
a hand-held therapy device comprising a housing having an end face, a side face, and a treatment surface extending between the end face and the side face, the treatment surface is inclined with respect to both the end face and the side face;
a phototherapy component configured to emit light for providing phototherapy treatment; and
a charging base configured to detachably connect to the hand-held therapy device, wherein the charging base comprises:
a base housing defining a slot for receiving the hand-held therapy device;
a shielding portion extending toward the hand-held therapy device and configured to cover the treatment surface during charging; and
a second charging coil to connect with a first charging coil disposed within the hand-held therapy device to wirelessly charge the hand-held therapy device.

11. The system of claim 10, wherein the first charging coil is arranged adjacent to the end face of the hand-held therapy device and the second charging coil is arranged adjacent to the bottom of the slot of the charging base.

12. The system of claim 10, wherein the charging base further comprises a second circuit board electrically connected to the second charging coil.

13. The system of claim 12, wherein the charging base further comprises a charging interface electrically connected to the second circuit board and configured to receive external power.

14. The system of claim 10, wherein the charging base further comprises a counterweight disposed within the base housing and spaced from the slot to enhance stability when the hand-held therapy device is inserted.

15. The system of claim 10, wherein the shielding portion is configured to prevent external light leakage during charging and to protect a light-transmitting area on the treatment surface from dust or scratches.

16. The system of claim 10, wherein the hand-held therapy device and the charging base are shaped and dimensioned such that the first and second charging coils are magnetically aligned when the device is inserted into the slot, thereby improving charging efficiency.

17. The system of claim 10, wherein the charging base further comprises a guiding structure configured to guide insertion of the hand-held therapy device into the slot and ensure proper positioning for charging.

18. A hand-held therapy device comprising:
a housing having an end face, a treatment surface, and a receiving cavity;
a phototherapy component disposed within the housing and oriented toward the treatment surface;
a mounting structure configured to detachably receive a care product container containing a cosmetic liquid;
a dispensing hole formed in the housing; and
a liquid-dispensing structure comprising a liquid flow channel disposed within the housing and extending between the mounting structure and the dispensing hole,
wherein the liquid flow channel is arranged to receive the cosmetic liquid from the care product container when the care product container is mounted to the mounting structure and to dispense the cosmetic liquid through the dispensing hole.

19. The hand-held therapy device of claim 18, further comprising:
a second liquid flow channel disposed within the housing; and a selector switch positioned on a side wall of the housing and movable among at least three positions,
wherein a first position closes both the liquid flow channel and the second liquid flow channel,
a second position opens only the liquid flow channel to allow dispensing from a first compartment of the care product container, and
a third position opens only the second liquid flow channel to allow dispensing from a second compartment of the care product container.

20. The hand-held therapy device of claim 19, wherein the liquid flow channel terminates at an outlet on the end face of the housing and the second liquid flow channel terminates at an outlet on the treatment surface of the housing to prevent cross-contamination between different cosmetic liquids.
